(19) United States
(12) Patent Application Publication
(10) Pub. No.: US 2011/0212948 A1
(43) Pub. Date: Sep. 1, 2011

(54) TREATMENT OF NEONATE FOALS WITH MELOXICAM

(76) Inventors: Giuseppe Pippia, Gladesville (AU); Sharanne Raidal, New South Wales (AU)

(21) Appl. No.: 13/062,372

(22) PCT Filed: Jul. 10, 2009
(86) PCT No.: PCT/AU09/00901

§ 371 Date: May 17, 2011

(30) Foreign Application Priority Data
Sep. 5, 2008 (AU) 2008904611

(51) Int. Cl. A61K 31/5415; C07D 417/12

(52) U.S. Cl. 514/226.5; 544/49

(57) ABSTRACT

A method of administering meloxicam containing compositions to neonate foals of 6 weeks of age or less is provided. The inventors have discovered that foals of 6 weeks of age or less can have meloxicam administered at a dosage of 0.6 mg/kg without any negative or adverse reaction by the foal. The method also provides a dosing regimen for obtaining first peak plasma concentrations of approximately 800 ng/ml, average plasma concentration levels of 200 ng/ml for 6 hours after a single dose, and sustained plasma concentration levels of 100 ng/ml with twice daily dosing.

### TECHNICAL FIELD

This invention relates to methods of providing meloxicam therapy to a neonate foal of 6 weeks or less and compositions for use in such methods.

### BACKGROUND ART

Non-steroidal anti-inflammatory drugs (NSAID's) are therapeutic agents with analgesic, antipyretic and anti-inflammatory effects. Most NSAIDs act as non-selective inhibitors of the enzyme cyclooxygenase, inhibiting both the cyclooxygenase-1 (COX-1) and cyclooxygenase-2 (COX-2) isoenzymes.

Some NSAID's selectively target the COX-2 enzyme over the COX-1 enzyme. These selective COX-2 inhibitors, of which meloxicam is a member, are generally less damaging to the subject patient's gastrointestinal tract than COX-1 type NSAID's. Notwithstanding that COX-2 directed NSAID's are less damaging to the gastrointestinal tract of the subject, COX-2 inhibitors such as meloxicam can still cause gastrointestinal irritation (vomiting, diarrhea and ulceration), and toxicity to the liver and kidney.

Furthermore, it is known that there are marked differences in drug pharmacokinetics between newborn and adult mammals. The pharmacokinetic alterations during the maturation process are related to changes in the pattern of absorption, distribution, metabolism and renal excretion. Delayed elimination of pharmaceutical active constituents and metabolites, including NSAID's can be due to various factors including underdeveloped renal function or immature metabolism of the drug itself. Unfortunately, as is often the case, an effective active constituent in an adult may in fact be toxic to a newborn or young mammal.

Given the uncertainty surrounding the safety of drug metabolism in newborn mammals, many compositions incorporating active constituents such as meloxicam have not been used in the absence of an indication that to do so would be safe.

Meloxicam is known to be effective in the treatment of pain including colic, fever and inflammation in adult horses. It is particularly effective as it can be delivered in a once a day dose due to its long half life in the plasma of the subject. However, meloxicam's long plasma half life and narrow range of therapeutic plasma concentrations in adult horses meant that previously it was not considered appropriate for use in the treatment of foals 6 weeks of age or younger. This is because if elimination of meloxicam from the foal is compromised due to the immaturity of the foal's renal system, high plasma levels of meloxicam may result in adverse or side effects including toxicity, organ damage and death.

Injuries to the foal occurring during foaling are common. In a significant amount of foalings the foal sustains injuries to its forelegs if they impact the uterus or ground during birth. Unfortunately, injuries sustained by the foal during the foaling process are by and large untreatable as there are no known, safe and efficacious analgesic and/or anti-inflammatory therapeutic agents that can be used in foals of less than 6 weeks of age.

It is an object of the present invention to provide NSAID compositions and methods for using them in the treatment of foals of 6 weeks of age or less.

### DISCLOSURE OF INVENTION

Surprisingly, it has been found that a meloxicam containing composition may be used for the treatment of foals of 6 weeks of age or less. It was considered unlikely that foals of this age would be able to tolerate, metabolise and excrete meloxicam due to their immature renal system.

Even more surprisingly however, it has been found that foals up to 6 weeks of age clear meloxicam from their bloodstream at twice the rate of adult horses and that repeated dosing of these young foals seemed to cause no adverse effects as measured by various assays including visual inspection of the gastrointestinal tract.

Accordingly, for the first time, it is now possible to administer meloxicam containing compositions to neonatal foals up to 6 weeks of age and obtain meloxicam concentrations in plasma, where this pharmacological effect can be considered safe.

According to a first aspect of the invention there is provided a method of administering meloxicam to a foal of 6 weeks of age or less, the method comprising administering an effective amount of a meloxicam containing composition that is sufficient to provide a first peak meloxicam plasma concentration of about 650 ng/mL to 1500 ng/mL about 30 to 60 minutes after providing the dose.

Preferably the administering of an effective amount of a meloxicam containing composition is sufficient to provide a first peak meloxicam plasma concentration of 800 ng/mL.

Preferably the administering of an effective amount of a meloxicam containing composition is sufficient to provide a meloxicam plasma level of 200 ng/mL for a period of six hours after the dose.

Preferably the meloxicam containing composition is administered at the rate of 0.6 mg/kg of meloxicam.

Preferably the method further comprises providing a second daily dose of the meloxicam containing composition that provides an average sustained meloxicam plasma concentration in the foal of at least 100 ng/mL.

Preferably the meloxicam containing composition is administered at the rate of 0.6 mg/kg of weight of the foal.

Preferably the composition contains 12% (w/v) of glycerol and 1.2% (w/v) of meloxicam in a liquid suspension.

More preferably the composition further comprises 0.5% (w/v) xantham gum to suspend the meloxicam.

Still more preferably the composition further comprises 0.14 (w/v) citric acid and 1.54% sodium dihydrogen phosphate (w/v).

Preferably the composition comprises sweeteners taken from the list of glycerol, xylitol, sodium saccharin and sorbitol.

More preferably the composition comprises 12% (w/v) glycerol, 14% (w/v) xylitol, 0.1% (w/v) sodium saccharin and 18% (w/v) sorbitol.

According to a second aspect of the invention there is provided a dosing regimen to obtain average meloxicam plasma concentrations of 650 ng/mL to 1500 ng/mL in neonate foals 6 weeks of age or less 30-60 minutes after administration that comprises administering to the foal a dose of meloxicam containing composition to the foal at the rate of at least 0.6 mg of meloxicam per kg of weight of the foal in a single dose.

According to a third aspect of the invention there is provided a dosing regimen to obtain average meloxicam plasma concentrations of 800 ng/mL in neonate foals 6 weeks of age or less 30-60 minutes after administration that comprises administering to the foal a dose of meloxicam containing composition to the foal at the rate of at least 0.6 mg of meloxicam per kg of weight of the foal in a single dose.

According to a fourth aspect of the invention there is provided a dosing regimen to obtain meloxicam plasma concentrations of 200 ng/mL for 6 hours in neonate foals 6 weeks of age or less that comprises administering to the foal a dose of meloxicam containing composition to the foal at the rate of at least 0.6 mg of meloxicam per kg of weight of the foal in a single dose.

According to a fifth aspect of the invention there is provided a dosing regimen to obtain sustained meloxicam plasma concentrations of 100 ng/mL in neonate foals 6 weeks of age or less that comprises administering to the foal dose of meloxicam containing composition to the foal at the rate of at least 0.6 mg of meloxicam per kg of weight of the foal, twice daily.

### BRIEF DESCRIPTION OF THE DRAWINGS

In order that the invention may be more readily understood and put into practical effect, reference will now be made to the accompanying drawings in which:—

FIG. 1 is a graph of plasma meloxicam concentration against hours post treatment for ten different foals,

FIG. 2 is a graph of plasma meloxicam concentration against hours following a single dose to 10 separate foals,

FIG. 3 is a graph of plasma meloxicam concentration against time of collection of plasma samples over 14 days for foals 6, 7, 8 and 9,

FIG. 4 is a graph of plasma meloxicam concentration against time of collection of samples every 12 hours for 16 days, pre and post treatment

FIG. 5 is a graph of plasma meloxicam concentration against time of collection of samples for single doses and multiple doses,

FIG. 6 is a graph of Body Weight against time of collection of samples for treated and control foals over 17 days,

FIG. 7 is a graph of Haemoglobin concentration against time of collection of samples for treated and control foals,

FIG. 8 is a graph of PCV concentration against time of collection of samples for treated and control foals,

FIG. 9 is a graph of Glucose concentration against time of collection of samples for treated and control foals,

FIG. 10 is a graph of Gamma glutamate transferase (GGT) concentration against time of collection of samples for treated and control foals,

FIG. 11 is a graph of Urea concentration against time of collection of samples for treated and control foals,

FIG. 12 is a graph of Creatinine concentration against time of collection of samples for treated and control foals,

FIG. 13 is a graph of Protein concentration against time of collection of samples for treated and control foals,

FIG. 14 is a graph of Albumin concentration against time of collection of samples for treated and control foals,

FIG. 15 is a photograph of the oesophageal entrance of a foal showing normal showing normal squamous mucosa pre-treatment,

FIG. 16 is a photograph of the margo plicatus of a control foal,

FIG. 17 is a photograph of the margo plicatus of a treated foal on Day 2 of the multiple dose study,

FIG. 18 is a photograph of normal margo plicatus on Day 7 of the multiple dose study,

FIG. 19 is a photograph of margo plicatus of a foal on day 14 of the multiple dose study,

FIG. 20 is a photograph of margo plicatus exhibiting erythemia,

FIG. 21 is a photograph of superficial erosions of squamous mucosa near margo plicatus of the same treated foal depicted in FIG. 20 on day 7 of the multiple dose study, and

FIG. 22 is a photograph of margo plicatus of the same foal show in FIGS. 20 and 21 following 14 days of treatment with meloxicam.

### MODES FOR CARRYING OUT THE INVENTION

Throughout the specification the following words are provided with the following meanings:

Meloxicam: Unless the context or text of the specification specifically provides otherwise, a reference to a meloxicam containing composition is a reference to a composition that contains either meloxicam in its free acid form or as a salt with a suitable anion such as sodium, potassium, meglumine, or ammonium anions. Further the meloxicam when in the free acid form, can be dissolved into solution for peroral or intravenous injection using either aqueous or polar solvents, or a combination of aqueous and polar phases in a microemulsion/liposomal preparation. Alternatively the meloxicam can be provided in a solid form whether suspended in a liquid to form a liquid suspension or paste, or pressed into a solid oral dosage form including tablets, granules, pellets or capsules.

BID:—twice daily dosing.

PCV: Packed Cell Volume

GGT: Gamma Glutamyl Transferase

BAR:—bright, alert, responsive.

Pharmaceutically acceptable additives include any of buffers, gelling agents, preservatives, oils, antioxidants, emulsifiers, solubilisers, foam forming agents, isotonic agents, viscosity enhancers and/or thickeners, preservatives, and buffers.

An aqueous based composition will now be illustrated by Example 1. However, it is expressly pointed out that the Examples provided are intended solely as an illustration and should not be regarded as restricting the invention.

### Example 1

### Liquid Suspension Composition

The liquid oral suspension of the present invention is comprised of the ingredients listed in Table 1.

TABLE 1

| | Percentage | Standard Amount |
|---|---|---|
| Ingredient | (w/v) | (g) |
| Meloxicam | 1.20% | 120.00 |
| Glycerol | 12.00% | 1200.00 |
| Xylitol | 14.00% | 1400.00 |
| Sodium Benzoate | 0.10% | 10.00 |
| Citric Acid | 0.14% | 14.00 |
| Sodium dihydrogen | 1.54% | 154.00 |
| phosphate | | |
| Sodium Saccharin | 0.10% | 10.00 |
| Xanthan gum | 0.50% | 50.00 |
| Sorbitol | 18.00% | 1800.00 |
| Water | 66.42% | 6642.00 |
| Total | 114.00% | 11400.00 |

The following steps were taken to formulate the liquid oral suspension composition containing meloxicam at a concentration of 12 mg/ml from the ingredients listed in Table 4.

In a suitable vessel collect the Water. To the water carefully add the Sodium Benzoate, Citric Acid, Sodium dihydrogen phosphate, Sodium Saccharin and Xylitol, mix to dissolve. Add the Xanthan gum and mix until completely hydrated. Next add the Sorbitol and Glycerol, mix until uniform. Transfer the bulk to the closed mixing vessel and continue mixing. Mix until a homogenous, smooth, lump-free product is obtained.

### Example 2

### Single Dosage Plasma Concentration Study

Ten healthy thoroughbred foals were recruited for this study. Foaling was supervised for each foal, and each was under veterinary supervision from the time of birth until recruitment into the study. All foals were healthy at the time of study; one foal was still receiving antibiotics for treatment of septic physitis of the distal femur. Mean age at the commencement of the study was 11 days (range 2 to 23 days) and mean body weight was 71.9 kg (range 53.5 to 96.5 kg).

Mares and foals were boxed on the day prior to the study, and foals underwent veterinary examination. Foals were sedated (xylazine 0.5-1.1 mg/kg, diazepam 0.05-0.18 mg/kg IMI) for placement of intravenous catheters in the left or right jugular vein on the day prior to treatment.

A single oral dose of 0.6 mg/kg of meloxicam, delivered as a 12 mg/mL suspension formulated as provided in Example 1, was given to each foal at 8 am on the day of treatment, following collection of pre-treatment blood samples for pharmacology and clinical pathology. Blood samples were withdrawn at 1, 1.5, 2, 2.5, 3, 4, 5, 6, 7, 8, 9, 10, 12, 16, 24, 36, 48, 72 and 96 hours following treatment. On the basis of initial results, obtained from the first 4 foals, subsequent post-treatment samples were obtained 0.5, 1, 1.5, 2, 2.5, 3, 4, 5, 6, 7, 8, 9, 10, 12, and 24 hours following treatment. All samples were kept on ice until separation of plasma by centrifugation, within 4 hours of collection. Plasma samples were stored frozen at −20° C. prior to analysis. Clinical pathology samples were repeated 24 hours after the administration of the drug; physical examination was performed twice daily for 36 hours and thereafter the foals were monitored daily as before the trial.

Plasma meloxicam concentrations following the administration of a single dose of meloxicam oral suspension (0.6 mg/mL) were determined by Ultra Performance Liquid Chromatography (UPLC) with UV detection, using piroxicam as an internal standard followed by protein denaturing using acetonitrile. A linear relationship between detector response and drug concentrations from 40 to 4000 ng/mL was demonstrated graphically and using regression analysis. The method limit of detection (10 ng/mL) was set by the addition of three times the standard deviation of the blank plasma extract signal to the blank plasma extract signal at the retention time of meloxicam. The limit of quantitation (20 ng/mL) was set at a value of 7 σ plus the blank plasma signal. The selectivity of this method for meloxicam was demonstrated by comparison of various chromatograms. The meloxicam response at 355 nm was considered to be free of matrix interferences. Method precision was assessed by replicate analyses of six replicate assays performed on fortified plasma samples from four foals containing incurred meloxicam (20-1500 ng/mL). Coefficients of variation for replicate analyses were considered within limits set for this plasma study (<3%).

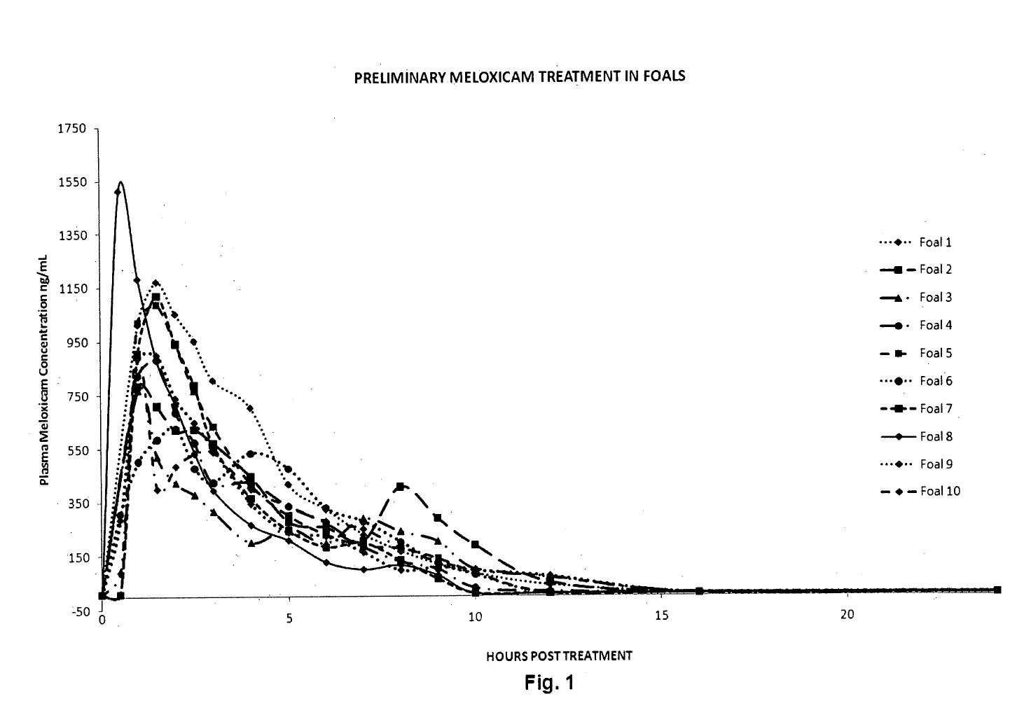

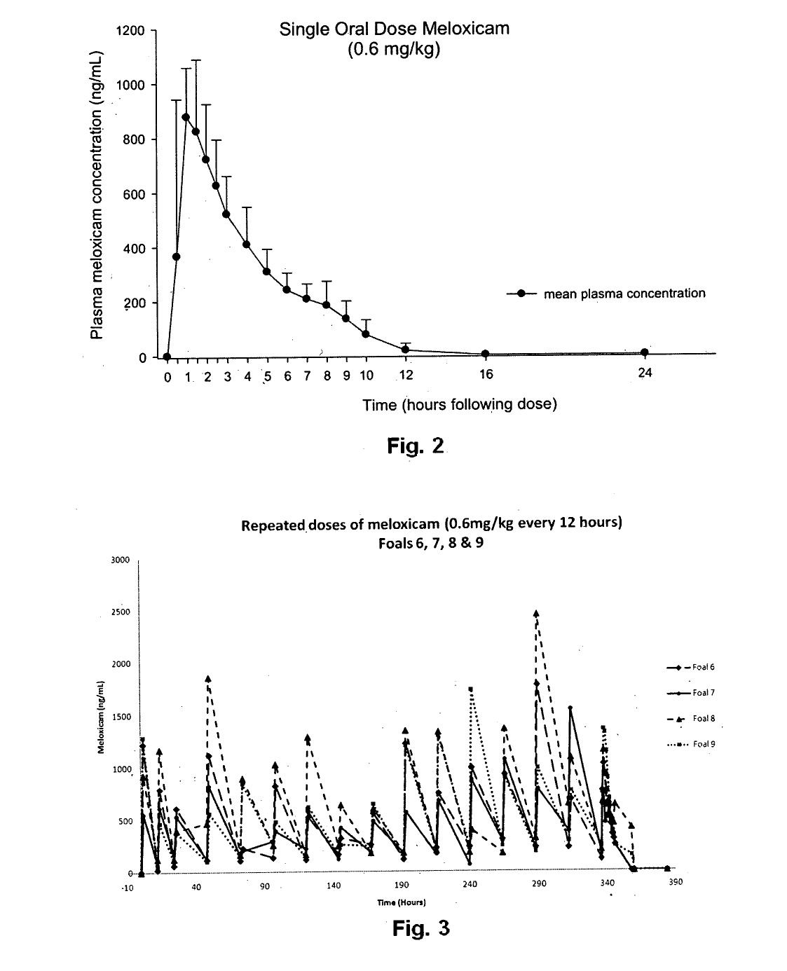

Plasma meloxicam concentrations from all foals are shown in FIG. 1 and mean plasma concentrations in FIG. 2. Maximum plasma concentrations were reported as observed. Plasma meloxicam concentration versus time curves were individually subjected to noncompartmental linear regression analysis using commercial software (PK Solutions, Summit Research Services, Montrose, Colo. 81401; www.SummitPK.com) to determine area under curve (AUC), time to maximum serum concentration (Tmax), elimination half life (T½β), apparent oral clearance and apparent volume of distribution. Apparent oral clearance and apparent volume of distribution were determined because meloxicam has not been administered intravenously to foals and the bioavailability (F) is not known in foals. Reports in adult horses (Toutain et al 2004) suggest bioavailability of between 84 and 100%, but bioavailability is generally reduced in neonates compared to adults (Bartelink et al 2006). Bioavailability was, therefore, assumed to be 90%.

Maximum plasma concentration (Cmax) was 974.1±254.0 ng/mL (range 627.1-1511.9 ng/mL) and time to maximum plasma concentration (Tmax) was 1.3±0.4 h (range 0.5-2.0 h). Mean elimination half-life was 2.8±0.9 hours; apparent oral clearance was 141.6±17.8 mL/kg/h and apparent volume of distribution was 578.9±235.0 mL/kg. These results are tabulated against similar results from adult horses in Table 2.

TABLE 2
Comparative results from current study and other reports in adult horses. Where available, results are
given as mean ± SD and range.

| | | Bioequivalence | Bioequivalence | | | Toutain | | |
|---|---|---|---|---|---|---|---|---|
| | | study | study | Little | Sinclair | and | | |
| Refer- | Current | (CML039) | (CML039) | et al | et al | Cester | Toutain et al | Lees et al |
| ence: | study | Test | Reference | 2007₁ | 2006₁ | 2004₂ | 2004b₃ | 1991₁ |
| Cmax | 974.1 ± | 917.4 ± | 777.5 ± | 9780 ± | | | 2580 ± | 9230 ± |
| (ng/mL) | 254.0_{(627.1-1511.9)} | 120.8_{(480.1-1119.0)} | 336.1_{(297.3-1474.6)} | 1000 | | | 580₍₁₄₈₀₋₃₄₆₀₎ | 1000₍₅₈₀₀₋₁₂₈₀₀₎ |
| (oral | | | | | | | | |
| admin) | | | | | | | | |
| Tmax | 1.3 ± | 2.6 ± | 3.8 ± | | | | 1.5 ± | |
| (h) (oral | 0.4 _{(0.5-2.0)} | 1.9_{(1.5-8)} | 2.8_{(1.5-10)} | | | | 1.07_{(1.0-4.0)} | |
| admin) | | | | | | | | |
| Elimi- | 2.8 ± | 9.4 ± | 10.2 ± | | | | 8.5 ± | 2.7 ± |
| nation | 0.9_{(1.9-3.6)} | 3.1 _{(5.2-14.3)} | 3.2_{(5.8-17.1)} | 4.07 ± | | (3.95-6.17)₄ | 3.0_{(5.1-14.5)4} | 0.44 |
| half- | | | | 1.07 | | | | |
| life (h) | | | | | | | | |
| Clear- | 141.6 ± | 57.2 ± | 58.8 ± | 19.8 ± | 34.7 ± 9.25 | (72-91)₅ | 34 ± | 41.9 ± |
| ance | 17.5_{(104.8-164.3)} | 18.2_{(36.1-96.6)} | 16.0_{(32.4-97.9)} | 8.4 | | | 5.7₍₂₄₋₄₁₎₅ | 2.85 |
| (mL/kg/h) | | | | | | | | |
| Area | 3870.3 ± | 11281.0 ± | 10752.4 ± | 33440 ± | | | | 14530 ± |
| under | 543.3_{(3221.3-5150.4)} | 3238.3_{(6135.0-16658.7)} | 2944.7_{(6041.9-18333.3)} | 10580 | | | | 800 |
| curve | | | | | | | | |
| (ng/mL/h) | | | | | | | | |
| Bioavail- | | | | | | | 95.9 ± | |
| ability | | | | | | | 13.2_{(84.0-117.1)} | |
| (fed) | | | | | | | | |
| Bioavail- | | | | | | | 85.3 ± | |
| ability | | | | | | | 19.4_{(63.4-126.7)} | |
| (fasted) | | | | | | | | |

As is evident from FIGS. 1 & 2 and Table 2, the administration of a single dose of 0.6 mg/kg meloxicam to foals resulted in a peak serum concentration approximating 1000 ng/mL. This is similar to Cmax obtained from adult horses in earlier studies (Table 2), but less than the value achieved following IV administration (Lees et al 1991, Toutain et al 2004, Little et al 2007). Median effective plasma concentrations of 130 ng/mL and 195 ng/mL have been demonstrated for improvement of clinical lameness score and stride length, respectively (Toutain and Cester 2004). Moses et al (2001) demonstrated that higher concentrations (5 μg/mL) were effective in decreasing PGE2 production by LPS-challenged equine synovial explants. More recent studies (Beretta et al 2005) have demonstrated dose-dependent inhibition of COX-1 and COX-2 production in equine peripheral blood associated with meloxicam concentrations of 35.1 ng/mL and above.

Foals demonstrated rapid absorption of meloxicam following oral administration (Tmax<1.5 h). Surprisingly, foals demonstrated rapid clearance of meloxicam (142 mL/kg/h), higher than reported in any adult studies. Consequently, elimination half-life (2.8 hours) was less than that reported for adult horses. The reasons for this observation are unclear—renal drug clearance is typically slower in neonates due to immature renal function (Bartelink et al 2006). However, differences in volume of distribution (data not shown), related to differences in water distribution (the extra cellular fluid compartment of neonates is typically 80-90% of total body water, cf 55-60% in adults) and/or reduced protein binding (Strolin Benedetti and Baltes 2003), may affect excretion and contribute to the observed rapid clearance. On the basis of these results a treatment interval of 12 h was recommended for foals.

Physical examination of foals in the days following administration of the drug demonstrated no adverse effects. Routine haematology and serum biochemistry results before and 24 h following the administration of a single dose of meloxicam were available for 6 foals. Comparison of results obtained prior to and following medication by paired t-test demonstrated no significant differences, except for serum sodium (Na+) concentrations, which were significantly increased in post-treatment samples (P=0.005). This finding is unlikely to be of clinical significance.

On the basis of the results recorded in Table 3, it was concluded that there were no adverse effects associated with the administration of a single oral dose of meloxicam to foals. Due to more rapid clearance, the drug should be administered every 12 hours to foals (compared to every 24 hours for adult horses).

TABLE 3
Selected clinical pathology data from foals prior to (pre-Tx) and
24 h following (post-Tx) a single oral dose of meloxicam
(0.6 mg/kg). Serum sodium concentrations increased significantly,
but no changes were observed in other parameters.

| | Mean pre-Tx | Mean post-Tx | |
|---|---|---|---|
| | (±sem) | (±sem) | P |
| Red blood cells | 9.3 (±0.3) | 9.2 (±1.0) | 0.552 |
| (×1012/L) | | | |
| Haemoglobin(g/L) | 125 (±3) | 123 (±4) | 0.423 |
| PCV (%) | 36 (±1) | 35 (±1) | 0.415 |
| Platelet count | 301 (±46) | 291 (±47) | 0.543 |
| (×109/L) | | | |
| White cell count | 8.3 (±0.4) | 9.0 (±0.6) | 0.216 |
| (×109/L) | | | |
| Neutrophil count | 6.4 (±0.5) | 6.8 (±0.8) | 0.595 |
| (×109/L) | | | |
| Sodium (mmol/L) | 135 (±1) | 138 (±1) | 0.005* |
| Potassium | 4.0 | 4.0 | 0.688 |
| (mmol/L)* | | | |
| Glucose (mmol/L) | 9.8 (±0.7) | 8.8 (±0.6) | 0.094 |
| Urea (mmol/L) | 2.5 (±0.2) | 2.1 (±0.1) | 0.626 |
| Creatinine | 0.09 | 0.09 | 1.00 |
| (mmol/L)* | | | |
| Total serum | 54 (±2) | 55 (±3) | 0.175 |
| protein (g/L) | | | |
| Albumin (g/L) | 28 (±1) | 29 (±1) | 0.661 |

*Data not normally distributed - non-parametric statistics (signed rank test) used, median result reported.

### Example 3

Six foals were available for inclusion in this part of the study and were randomly assigned to treatment (four foals) or control groups (two foals). Foals in the treatment group received meloxicam 0.6 mg/kg by mouth every 12 hours (8 am and 8 pm) for 14 days; control foals received an equivalent volume of vehicle only at the same times. All foals were healthy at the beginning of the study, although one foal was receiving ongoing antibiotic treatment for suspected septic distal femoral physitits. Average age (24.3±7.5 days) and body weight (89.8±17 kg) of treated foals at the commencement of the study were not significantly different to control foals (24.0±4.2 days and 94.7±18.0 kg, respectively) (mean±standard deviation). To ensure foals were recruited into the study within 4 weeks of birth, the study was performed twice, with 2 treated and one control foal in each replicate.

Foals were examined twice daily for the duration of the study. Staff responsible for veterinary examination of foals were blinded to treatment. Blood was collected for determination of plasma meloxicam concentrations according to the schedule appearing in Table 4.

TABLE 4

| | |
|---|---|
| Day 0 | 0 h |
| | 2 h (peak) |
| | 12 h (trough) |
| | 14 h (peak) |
| Day 1 | 24 h (trough) |
| | 26 h (peak) |
| Day 2 | 48 h (trough) |
| | 50 h (peak) |
| Day 3 | 72 h (trough) |
| | 74 h (peak) |
| Day 4 | 96 h (trough) |
| | 98 h (peak) |
| Day 5 | 120 h (trough) |
| | 122 h (peak) |
| Day 6 | 144 h (trough) |
| | 146 h (peak) |
| Day 7 | 168 h (trough) |
| | 170 h (peak) |
| Day 8 | 192 h (trough) |
| | 194 h (peak) |
| Day 9 | 216 h (trough) |
| | 218 h (peak) |
| Day 10 | 240 h (trough) |
| | 242 h (peak) |
| Day 11 | 264 h (trough) |
| | 266 h (peak) |
| Day 12 | 288 h (trough) |
| | 290 h (peak) |
| Day 13 | 312 h (trough) |
| | 314 h (peak) |
| Day 14 | 336 h (trough) |
| | 337 h |
| | 338 h |
| | 339 h |
| | 340 h |
| | 341 h |
| | 342 h |
| | 343 h |
| | 344 h |
| | 345 h |
| | 346 h |
| | 348 h |
| Day 15 | 360 h |
| Day 16 | 384 h |

Peripheral blood samples were submitted for routine haematology and serum biochemistry at a commercial laboratory (Idexx Laboratories, Rydalmere, NSW) twice weekly (treatment days 0, 2, 6, 9, 13 and 16). Gastroscopy and urine analysis (Dip Stik and urine enzyme analysis1) were performed weekly on all foals.

Meloxicam was administered to foals in the treatment group daily at 8 am and 8 pm. Blood for ‘trough’ serum levels was collected from foals immediately prior to administration of morning treatment. ‘Peak’ plasma concentrations were determined from blood samples collected 2 hours following administration of the morning treatment (as per the schedule above).

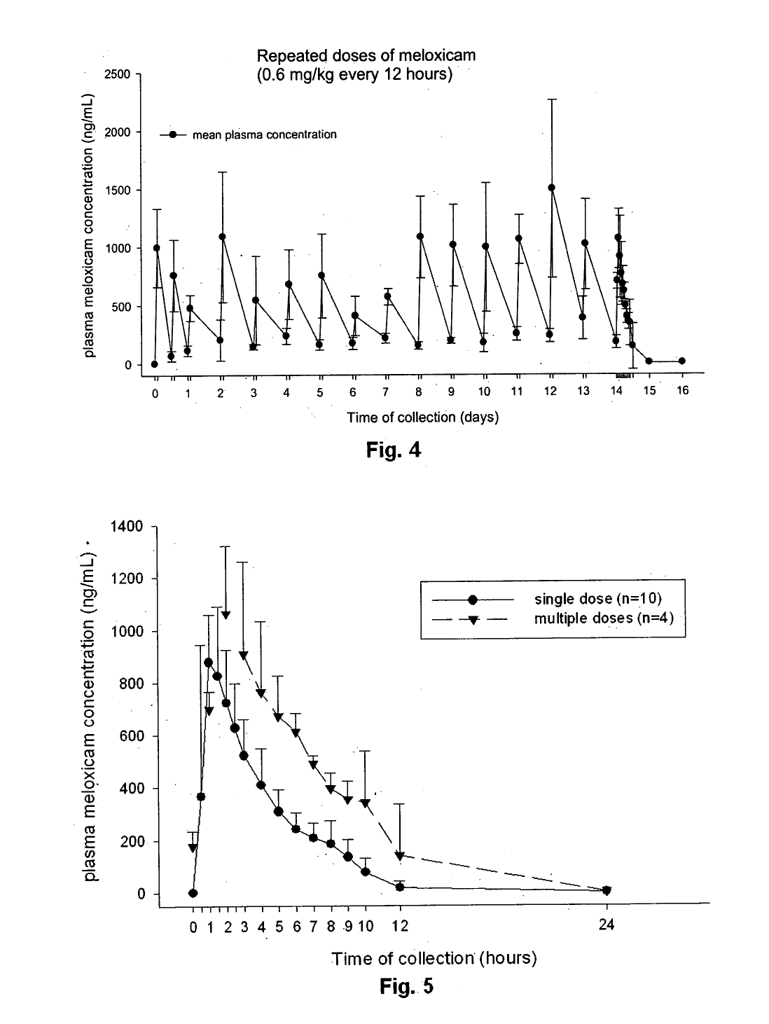

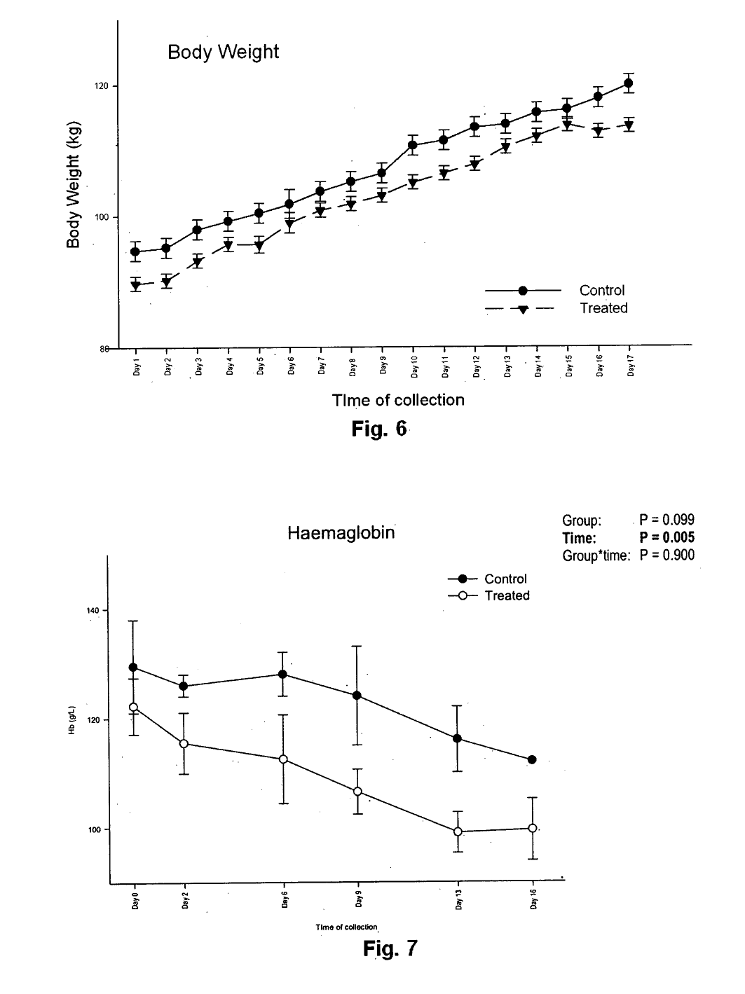

Raw data from all foals receiving meloxicam is shown in FIG. 3 and mean data in FIG. 4. Repeated dosing was associated with peak serum concentrations between 500 and 1200 ng/mL. The drug did not appear to accumulate in serum, and the excretion profile at the end of treatment was similar to that obtained following a single dose (FIG. 5), suggesting that drug excretion was unchanged by repeated administration.

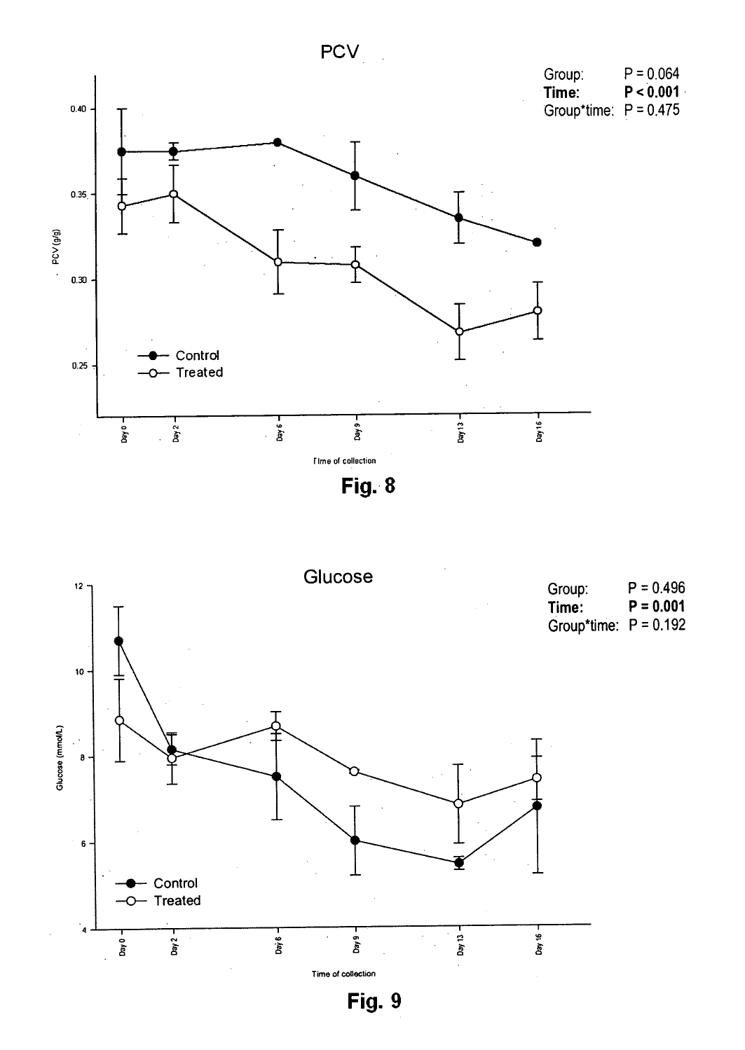

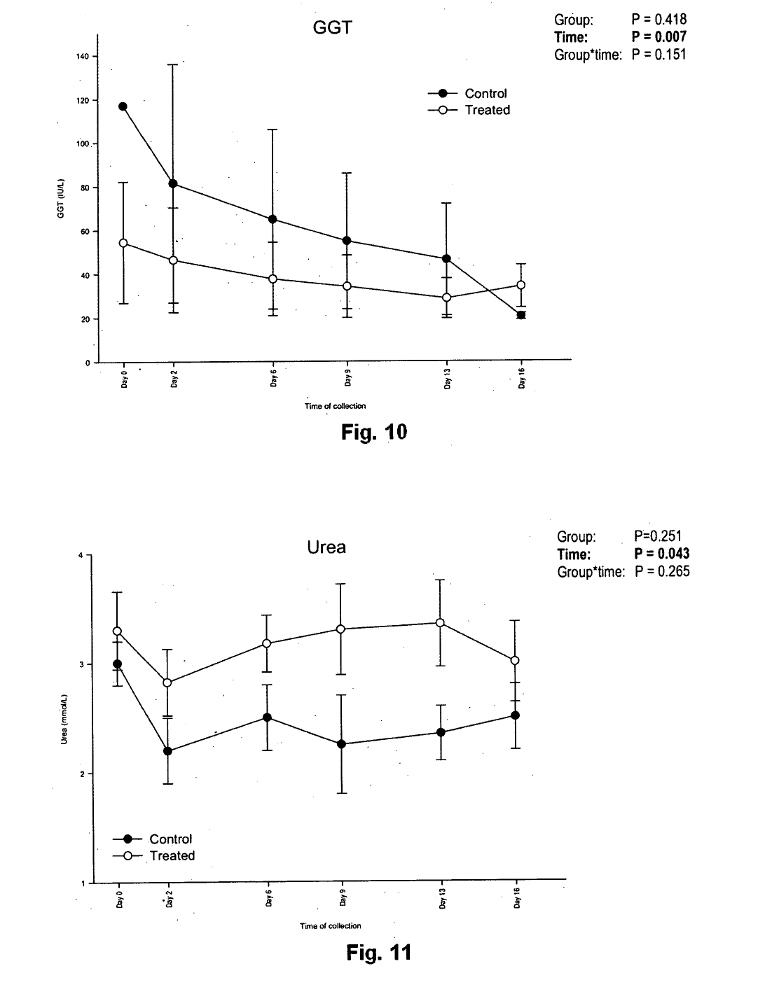

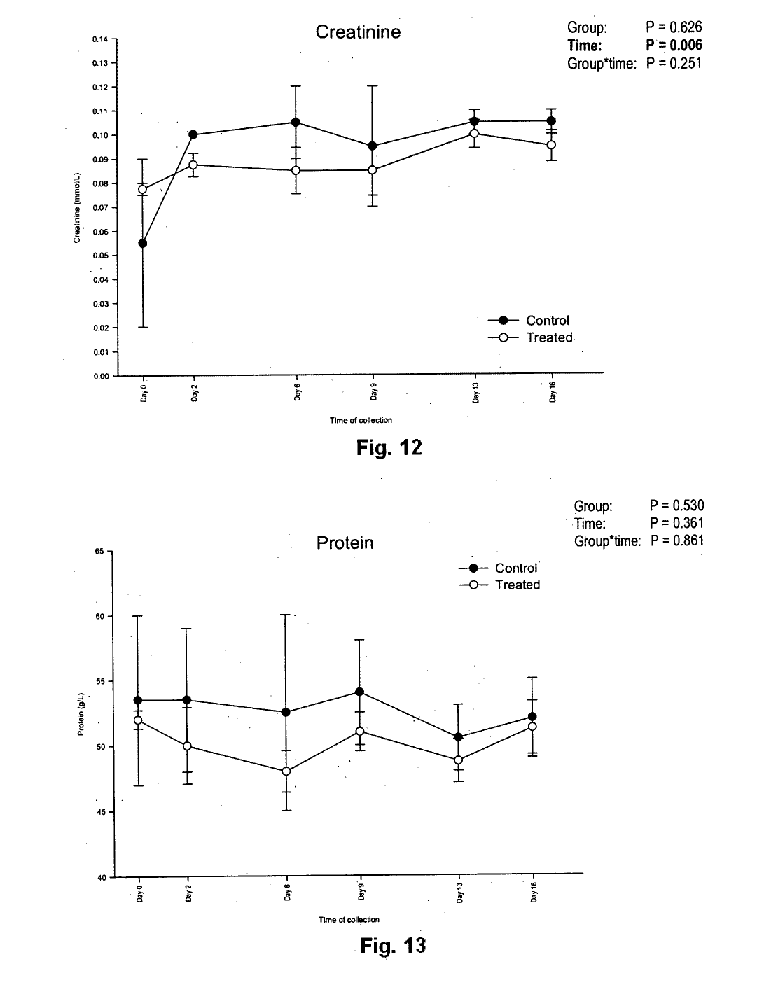

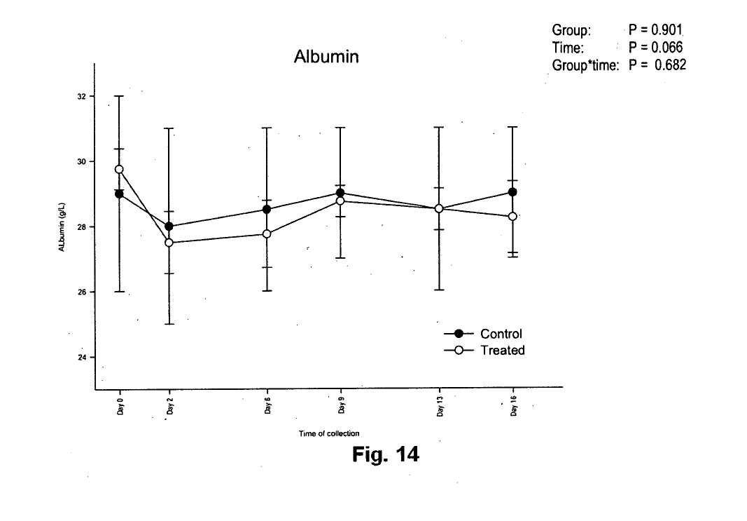

Results of haematology and serum biochemistry assays demonstrated no significant differences between treated and control foals, with the exception of significant differences in serum cholesterol concentrations between treated and control foals on days 2 and 6. This is likely to be a spurious finding, as the apparent difference between control and treated foals was evident at Day 0. Pooled results from both groups demonstrated a significant time of collection effect in some parameters (notably haemoglobin, PCV, glucose and GGT, FIGS. 7-10). Decreases in these parameters during the experimental period may reflect progressive acceptance of handling procedures by foals. Significantly, there was no evidence of changes in serum urea or creatinine concentrations (indicative of renal function, FIGS. 11 and 12), nor in serum total protein or albumin concentrations (protein loss may occur with renal or gastrointestinal damage, FIGS. 13 and 14).

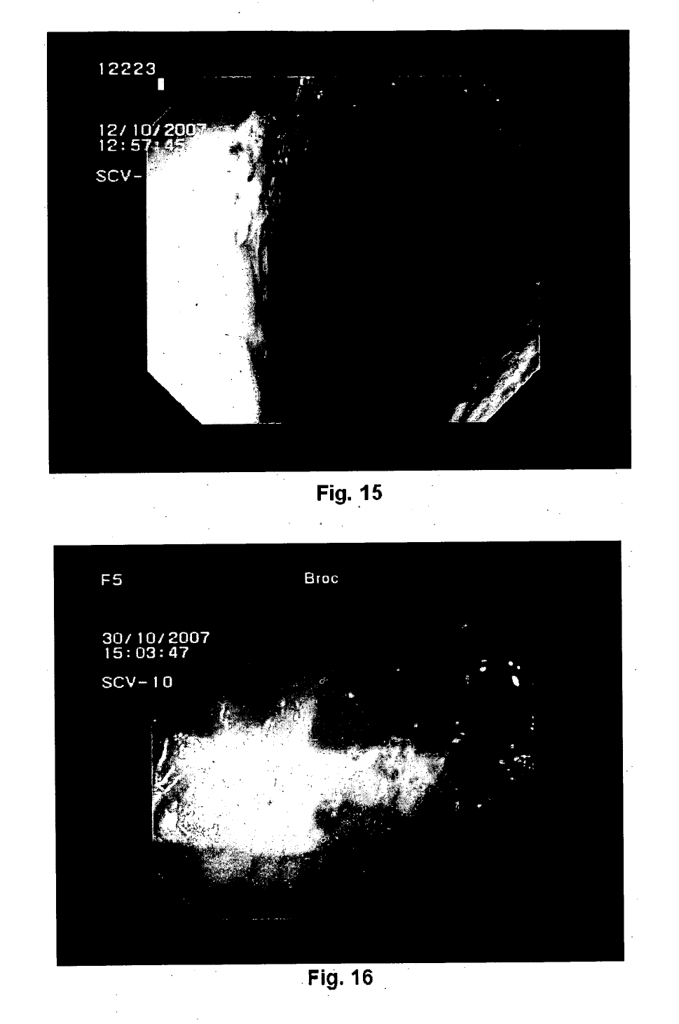

Gastroscopic examination of all foals failed to identify evidence of significant gastric mucosal damage in treated or control foals. Representative images from treated and untreated foals are shown in FIGS. 15-22.

FIG. 15 shows the view of oesophageal entrance showing normal squamous mucosa in a healthy foal in the single dose study (F5, pre-treatment).

FIG. 16 shows an inverted view of margo plicatus (greater curvature) of control foal at the commencement of the multiple dose study. There is mild hyperkeratosis of the squamous mucosa (bottom) and a ‘skin’ of clotted milk.

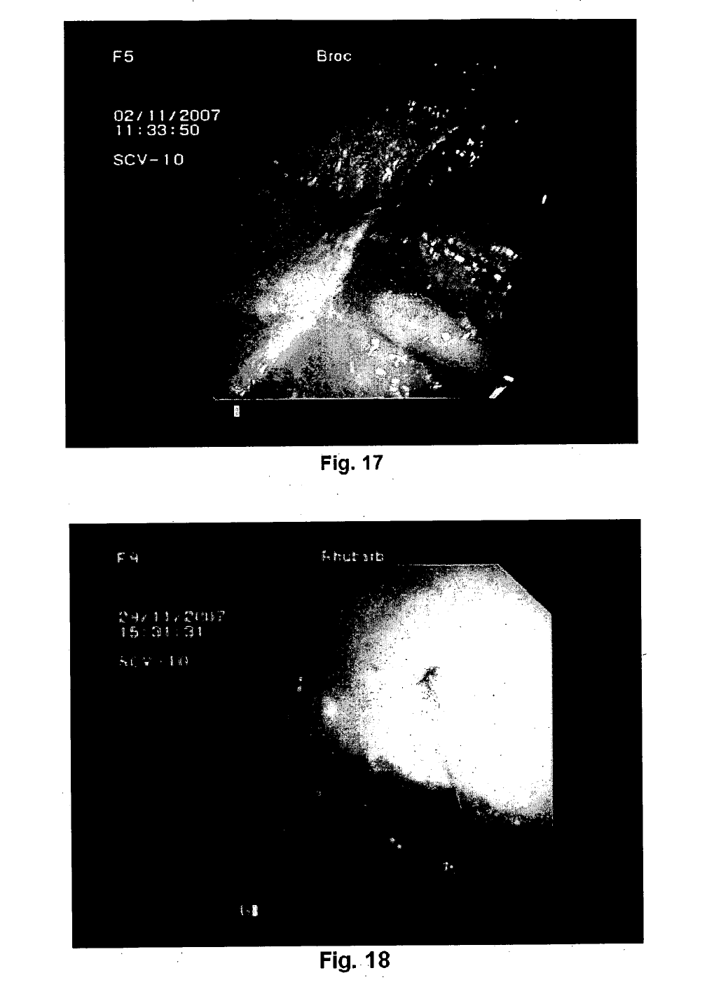

FIG. 17 shows margo plicatus (greater curvature) of control foal depicted in FIG. 16 on Day 2 of the multiple dose study showing healthy squamous and glandular mucosae.

FIG. 18 shows the normal margo plicatus (greater curvature) of a treated foal on Day 7 of the multiple dose study.

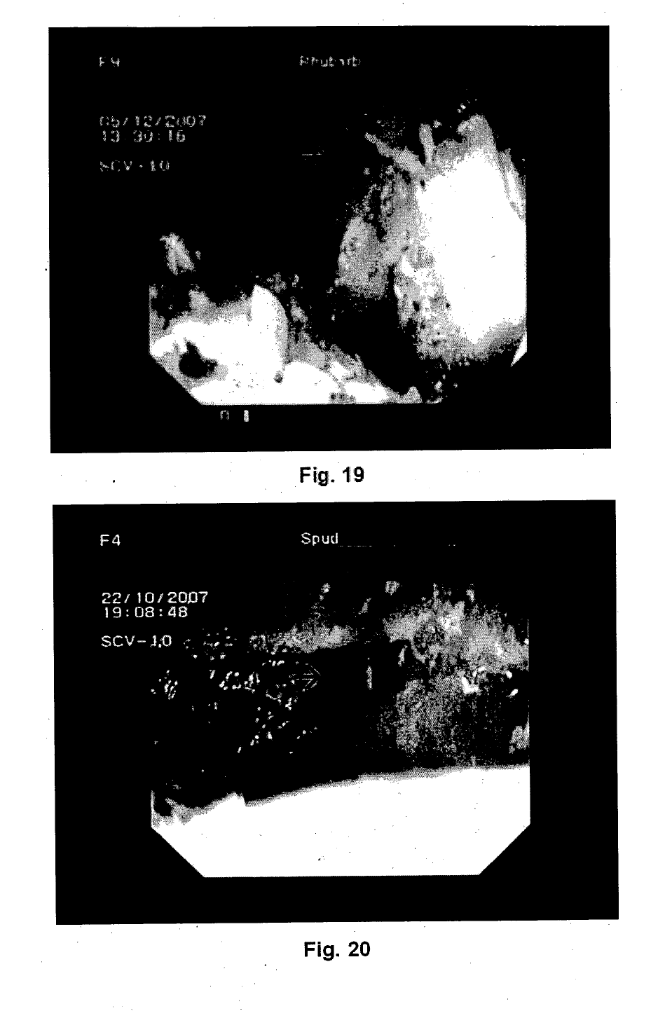

FIG. 19 shows the Margo plicatus from same foal as depicted in FIG. 18 on Day 14 of multiple dose study. A small, well circumscribed and very superficial ulcer is evident (arrow).

FIG. 20 shows the Margo plicatus (greater curvature) of a healthy foal (F8) prior to administration of a single dose of meloxicam. A small, well circumscribed area of erythemia is shown in this photograph.

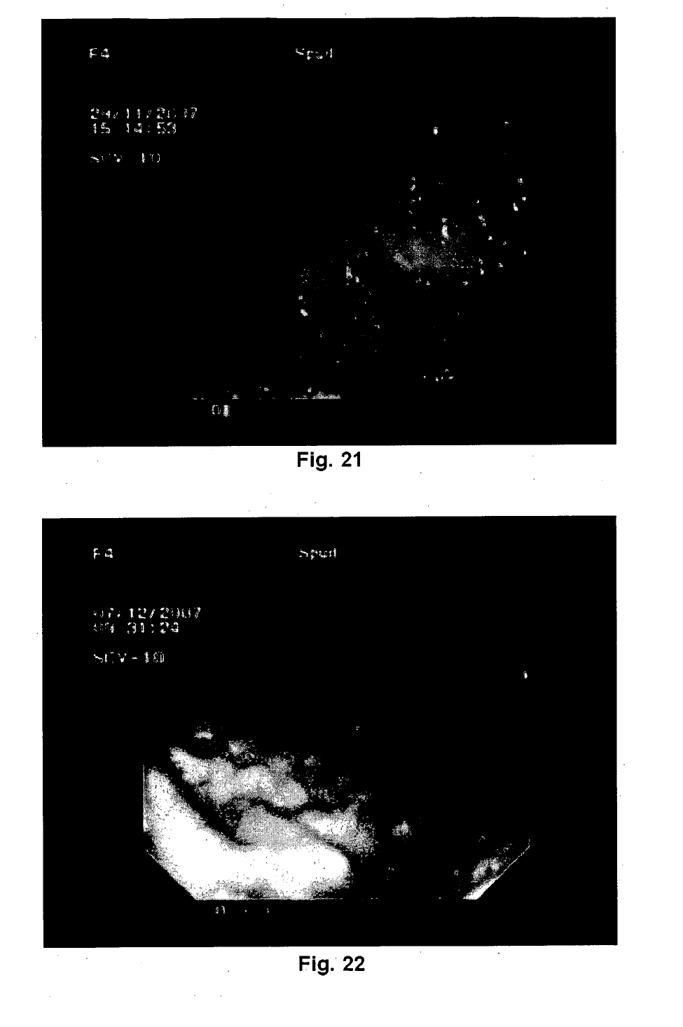

FIG. 21 shows superficial erosions of squamous mucosa near margo plicatus of the same (treated) foal as depicted in FIG. 20 on Day 7 of multiple dose study. Ulceration is more extensive and largely restricted to squamous mucosa. FIG. 22 depicts margo plicatus (greater curvature) of the same foal as FIGS. 20 and 21 following 14 days of treatment with meloxicam. There is no evidence of ongoing mucosal ulceration despite continued treatment and box confinement.

Faecal occult blood testing using guaiac-based slides (Hemoccult Sensa, Beckman Coulter Australia Pty Ltd, Gladesville, NSW) was evaluated for the detection of gastrointestinal bleeding in treated and control foals. This technique has been reported as a sensitive method for identifying gastric or right dorsal colon ulceration in adult horses (Pellegrini 2005). Faecal samples were collected on DSP days from all foals and processed according to the manufacturer's instructions. Tests were negative for blood (haemoglobin) on all occasions.

On the basis of these findings it was concluded that the administration of meloxicam to foals less than 6 weeks of age was not associated with adverse clinical changes in healthy foals and that the administration of meloxicam at a dose rate of 0.6 mg/kg by mouth every 12 hours achieved plasma meloxicam concentrations between 100 and 1000 ng/mL.

References made in this specification to other patents or scientific publications are not to be taken as an admission that said references are common general knowledge.

The present disclosure has been described relative to a preferred embodiment. Improvements or modifications that become apparent to persons of ordinary skill in the art only after reading this disclosure are deemed within the spirit and scope of the application. It is understood that several modifications, changes and substitutions are intended to be included in the scope of the present invention.

1. A method of administering meloxicam to a foal of 6 weeks of age or less, the method comprising administering an effective amount of a meloxicam containing composition that is sufficient to provide a first peak meloxicam plasma concentration of about 650 ng/mL to 1500 ng/mL about 30 to 60 minutes after providing the dose.

2. The method of claim 1 wherein the administering of an effective amount of a meloxicam containing composition is sufficient to provide a first peak meloxicam plasma concentration of approximately 800 ng/mL.

3. The method of claim 1 wherein the administering of an effective amount of a meloxicam containing composition is sufficient to provide a meloxicam plasma concentration of approximately 200 ng/mL for six hours.

4. The method of claim 1 wherein the meloxicam containing composition is administered at the rate of 0.6 mg/kg of meloxicam.

5. The method of claim 4 that further comprises providing a second daily dose of the meloxicam containing composition that provides an average sustained meloxicam plasma concentration in the foal of at least 100 ng/mL.

6. The method of claim 5 wherein the second daily dose of the meloxicam containing composition is administered at the rate of 0.6 mg/kg of weight of the foal.

7. The method of claim 6 wherein the composition is a liquid suspension of meloxicam in its free acid form that is taken orally.

8. The method of claim 7 wherein the liquid suspension contains 12% (w/v) of glycerol and 1.2% (w/v) of meloxicam in a liquid suspension.

9. The method of claim 8 wherein the liquid suspension further comprises 0.5% (w/v) xantham gum as a suspending agent.

10. The method of claim 9 wherein the liquid suspension further comprises 0.14 (w/v) citric acid and 1.54% sodium dihydrogen phosphate (w/v).

11. The method of claim 10 wherein the liquid suspension further comprises sweeteners taken from the list of glycerol, xylitol, sodium saccharin and sorbitol.

12. The method of claim 11 wherein the liquid suspension uses all of the following sweeteners: glycerol, xylitol, sodium saccharin and sorbitol.

13. The method of claim 11 wherein the sweeteners have the following concentrations in the liquid suspension: 12% (w/v) glycerol, 14% (w/v) xylitol, 0.1% (w/v) sodium saccharin and 18% (w/v) sorbitol.

14. A dosing regimen to obtain meloxicam plasma concentrations of 650 ng/mL to 1500 ng/mL in neonate foals 6 weeks of age or less 30-60 minutes after administration that comprises administering to the foal a dose of meloxicam containing composition to the foal at the rate of at least 0.6 mg of meloxicam per kg of weight of the foal in a single dose.

15. A dosing regimen to obtain meloxicam plasma concentrations of 800 ng/mL in neonate foals 6 weeks of age or less 30-60 minutes after administration that comprises administering to the foal a dose of meloxicam containing composition to the foal at the rate of at least 0.6 mg of meloxicam per kg of weight of the foal in a single dose.

16. A dosing regimen to obtain meloxicam plasma concentrations of 200 ng/mL for 6 hours in neonate foals 6 weeks of age or less that comprises administering to the foal a dose of meloxicam containing composition to the foal at the rate of at least 0.6 mg of meloxicam per kg of weight of the foal in a single dose.

17. A dosing regimen to obtain sustained meloxicam plasma concentrations of 100 ng/mL in neonate foals 6 weeks of age or less that comprises administering to the foal dose of meloxicam containing composition to the foal at the rate of at least 0.6 mg of meloxicam per kg of weight of the foal, twice daily.
